(19) United States
(12) Patent Application Publication
(10) Pub. No.: US 2023/0024982 A1
(43) Pub. Date: Jan. 26, 2023

(54) TOUCH-RELATED CONTAMINATION STATE DETERMINATIONS

(71) Applicant: Hewlett-Packard Development Company, L.P., Spring, TX (US)

(72) Inventors: MANOHAR LAL KALWANI, Pune (IN); Abhishek Ghosh, Spring, TX (US)

(21) Appl. No.: 17/583,830

(22) Filed: Jan. 25, 2022

(30) Foreign Application Priority Data
Jul. 19, 2021 (IN) 2021441032389

(51) Int. Cl. H04L 67/50; G06N 20/00; G06K 9/62

(52) U.S. Cl. CPC H04L 67/22; G06N 20/00; G06K 9/6262; A61L 2/00

(57) ABSTRACT

In an example, a non-transitory machine-readable storage medium may include instructions that, when executed by a processor of a computing device, cause the processor to receive device usage data associated with an electronic device. Further, instructions may be executed by the processor to determine a touch-related contamination state of a surface of the electronic device by applying a machine learning model to the device usage data. Furthermore, instructions may be executed by the processor to send an alert notification to the electronic device based on the touch-related contamination state.

### BACKGROUND

Electronic devices, such as desktop computers, laptop computers, point of sale systems, image forming apparatuses, or the like, may include a user interface that permits a user to interact with the electronic devices. An example user interface may include a keyboard, a touch screen display panel, a mouse, a touch pad, or the like. The user interface may enable users to input data into the electronic devices, for instance, either by touching a touch interface or by pressing keys. Such electronic devices may be shared by multiple users, for instance, in a public or private environment such as an enterprise, a hospital, a home, an educational establishment, or the like.

### BRIEF DESCRIPTION OF THE DRAWINGS

Examples are described in the following detailed description and in reference to the drawings, in which:

FIG. 1 is a block diagram of an example computing device including a non-transitory machine-readable storage medium, storing instructions to determine a touch-related contamination state of a surface of an electronic device;

FIG. 2 is a block diagram of an example computing device including a non-transitory machine-readable storage medium, storing instructions to determine a machine learning model from a set of machine learning models to estimate a touch-related contamination state;

FIG. 3A is a block diagram of an example electronic device, including a processor to output an alert notification including a recommended action to clean the electronic device;

FIG. 3B is a block diagram of the example electronic device of FIG. 3A, depicting additional features;

FIG. 4 is a block diagram of an example computing environment, depicting the example electronic device of FIG. 3A in communication with a computing device;

FIG. 5A is a flowchart illustrating an example process for training a machine learning model based on historical device usage data; and

FIG. 5B is a flowchart illustrating an example process for determining a touch-related contamination state of an electronic device by applying the trained machine learning model (e.g., of FIG. 5A) on real-time device usage data.

### DETAILED DESCRIPTION

Electronic devices, such as desktop computers, laptop computers, point of sale systems, image forming apparatuses, or the like may be used in different environments (e.g., an enterprise, a hospital, a home, an educational establishment, or the like). In such example environments, the electronic devices may be shared by multiple users. Further, the electronic devices may include a user interface (e.g., a keyboard, a touch screen display panel, a mouse, a touch pad, and/or the like) to permit users to interact with the electronic devices. The user interface may be hand operated, where the users touch keys, buttons, and/or touch screens with hand(s) and/or finger(s).

Consequently, contaminants present on the users hand, including dirt, debris, bacteria, germs, fungus, viruses, and other pathogenic microorganisms may transfer onto a surface of the electronic devices, for instance. In such a scenario, contamination and cross-contamination through the surface of the electronic devices may be a concern. The surface may be a mode of transmission of spreadable contaminants from one user to another user as the contaminants can stay active for significantly longer time on such surfaces (e.g., which are made up of a glass, plastic, and/or the like). For example, the keyboard may be a contaminated common-touch surface in hospitals, with a study showing approximately 62% contamination.

Some example electronic devices may include touch capacitive sensors arrayed over a surface (e.g., a touch surface) of the electronic device to detect user interaction such as finger resting, sliding, tapping, and/or pressing. Such detected user interactions may be used to determine the surface contamination. However, the array of touch capacitive sensors may consume significant amount of space, and hence may result in an increased size (e.g., a thickness) of the electronic devices. Also, the array of touch capacitive sensors may involve additional hardware and increased costs.

Examples described herein may provide a computing device that utilizes a machine learning model to determine a touch-related contamination state of a surface of an electronic device. The computing device may be a server that is communicatively connected to the electronic device via a network. The computing device may obtain historical device usage data (e.g., user login data, keyboard usage data, location data, user facial recognition data, user behavior data, and/or the like) associated with the electronic device. Further, the computing device may process the historical device usage data to generate a train data set and a test data set. Furthermore, the computing device may build a set of machine learning models with the train data set to determine the touch-related contamination state of the surface of the electronic device. Also, the computing device may test the trained set of machine learning models with the test data set. In addition, the computing device may select the machine learning model having a high accuracy from the set of trained and tested machine learning models to estimate the touch-related contamination state of the electronic device.

During operation, the computing device may receive real-time device usage data associated with the electronic device. Further, the computing device may determine a touch-related contamination state of the surface of the electronic device by applying the selected machine learning model to the real-time device usage data. Furthermore, the computing device may send an alert notification to the electronic device based on the touch-related contamination state. The alert notification may also include a recommended action/process to clean or sanitize the electronic device.

Thus, examples described herein may utilize the machine learning model to determine the touch-related contamination state of the electronic device. Further, prior to the usage of the electronic device, examples described herein may alert the users with instructions to proactively clean the electronic devices, thereby avoiding undue health problems.

In the following description, for purposes of explanation, numerous specific details are set forth in order to provide a thorough understanding of the present techniques. However, the example apparatuses, devices, and systems, may be practiced without these specific details. Reference in the specification to “an example” or similar language means that a particular feature, structure, or characteristic described may be included in at least that one example but may not be in other examples.

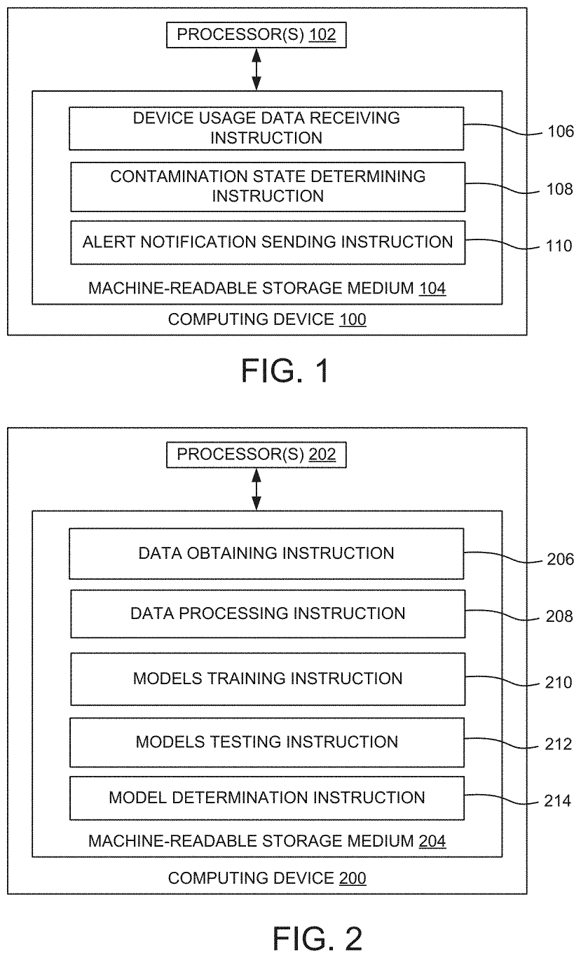

Turning now to the figures, FIG. 1 is a block diagram of an example computing device 100 including a non-transitory machine-readable storage medium 104, storing instructions to determine a touch-related contamination state of a surface of an electronic device. In an example, computing device 100 may be a server operating in a cloud computing system, a server configured in a Software as a Service (SaaS) architecture, or the like. An example electronic device may be a desktop computer, a laptop computer, a point-of-sale system, a smartphone, or any other device having a touch-based input surface.

Further, computing device 100 may be communicatively connected to the electronic device via a network. Example network can be a managed Internet protocol (IP) network administered by a service provider. For example, the network may be implemented using wireless protocols and technologies, such as Wi-Fi, WiMax, and the like. In other examples, the network can also be a packet-switched network such as a local area network, wide area network, metropolitan area network, Internet network, or other similar type of network environment. In yet other examples, the network may be a fixed wireless network, a wireless local area network (LAN), a wireless wide area network (WAN), a personal area network (PAN), a virtual private network (VPN), intranet, or other suitable network system and includes equipment for receiving and transmitting signals.

Computing device 100 may include a processor 102 and machine-readable storage medium 104 communicatively coupled through a system bus. Processor 102 may be any type of central processing unit (CPU), microprocessor, or processing logic that interprets and executes machine-readable instructions stored in machine-readable storage medium 104.

Machine-readable storage medium 104 may be a random-access memory (RAM) or another type of dynamic storage device that may store information and machine-readable instructions that may be executed by processor 102. For example, machine-readable storage medium 104 may be synchronous DRAM (SDRAM), double data rate (DDR), Rambus® DRAM (RDRAM), Rambus® RAM, and the like, or storage memory media such as a floppy disk, a hard disk, a CD-ROM, a DVD, a pen drive, and the like. In an example, machine-readable storage medium 104 may be a non-transitory machine-readable medium, where the term “non-transitory” does not encompass transitory propagating signals. In an example, machine-readable storage medium 104 may be remote but accessible to computing device 100.

Machine-readable storage medium 104 may store instructions 106-110. In an example, instructions 106 may be executed by processor 102 to receive device usage data associated with the electronic device, for instance, via the network. For example, the device usage data may include user login data, input device usage data, location data, user facial recognition data, user behavior data, or any combination thereof. In an example, the user login data may include audit log information that is used to track user sign-ins to the electronic device, user sign-ins to an application running on the electronic device, or the like. When users' log in to the electronic device, event logs may be generated and stored as the audit log information in the electronic device. The audit log information may be used to identify the users, differentiate the users from one another, and determine a number of users who have logged in to the electronic device.

The input device usage data may include usage data associated with an input device such as a mouse, a touchpad, a touchscreen, a keyboard, a joystick, or any combination thereof. In some examples, a keylogger may be used to record input device usage data associated with the electronic device. The keylogger may be a device or a computer program in the electronic device that is capable of capturing and/or storing input provided by the input device. For example, usage data of the keyboard may include keystrokes performed by the users operating and/or interacting with the electronic device. Similarly, usage data of the mouse may include information indicative of the mouse location, mouse movement, mouse button click event, and so on.

The location data may be used to determine a location of the electronic device. The location data may include global positioning system (GPS) information (e.g., latitude and longitude data) corresponding to the location of the electronic device. The location data may be captured using a sensor (e.g., a GPS sensor) in the electronic device. The captured location data can be fed to a geo location application programming interface (API) (e.g., a Google map API) to identify public or private spaces.

The user facial recognition data may include image data or video data captured via a camera (e.g., a two-dimensional camera, a three-dimensional camera, an infrared camera, or the like) of the electronic device. In another example, the user facial recognition data may include a set of points, edges, skin textures, and/or similar data that can be used to differentiate users of the electronic device. The user facial recognition data can be evaluated to identify the users, differentiate the users from one another, and determine a number of users who have accessed the electronic device. Thus, the user facial recognition data may be used to distinguish users from one another and to categorize the device usage data (e.g., the input device usage data) of the users.

The user behavior data may include the image data or the video data captured via the camera. The user behavior data can be evaluated to determine a user activity or a user behavior that can contaminate the surface of the electronic device. In an example, the user activity or the user behavior that can contaminate the surface may indicate whether the user is touching eyes, nose, mouth, food, or the like, while working on the electronic device. In another example, the user activity or the user behavior may indicate whether the user is coughing, sneezing, or the like, that can expel droplets onto the surface of the electronic device.

In an example, instructions to receive the device usage data may include instructions to receive the device usage data associated with the electronic device at a periodic interval or in response to a user login event to the electronic device (e.g., when a user logs in to computing device 100). Further, computing device 100 may receive the device usage data from the electronic device via an API call, for instance.

Instructions 108 may be executed by processor 102 to determine a touch-related contamination state of a surface of the electronic device by applying a machine learning model to the device usage data. The “machine-learning model” may refer to a computer representation that can be tuned (e.g., trained) based on inputs to approximate unknown functions. In particular, the term “machine-learning model” can include a model that utilizes methods to learn from, and make predictions on, known device usage data by analyzing the known device usage data to learn to generate outputs (e.g., touch-related contamination states) that reflect patterns and attributes of the known device usage data.

For instance, the machine learning model may be a supervised machine learning model that implements a classification method such as a random forest, XG boost, logistic regression, or the like. In other examples, the machine-learning model can include, but not limited to, a decision tree, support vector machine, Bayesian network, dimensionality reduction algorithm, artificial neural network, and deep learning. Thus, the machine-learning model makes high-level abstractions in the device usage data by generating data-driven predictions or decisions from the inputted device usage data.

In an example, the machine learning model may be applied to the device usage data to determine the number of users logged in to the electronic device, an area (e.g., a key, a touch surface, or the like) of the input device that is touched by a user, the device location where the electronic device is used (e.g., whether the electronic device is used in the public space or the private pace), the number of users accessed the electronic device, the user activity or the user behavior that can contaminate the surface of the electronic device, and the like. Further, the machine learning model may determine the touch-related contamination state of the surface of the electronic device based on the determined number of users, touched area, device location, user behavior, and the like.

In an example, the touch-related contamination state may include information indicating a contaminated area on the surface of the electronic device, a contamination level of the contaminated area, or a combination thereof. For example, the contaminated area on the surface of the electronic device may include a key or a set of keys touched by the users, a portion of the touchscreen touched by the user, a touchpad, or the like. Further, the contamination level may be an index value indicating a level of contamination (e.g., low, medium, and high) of the surface of the electronic device based on the device usage data.

In an example, when the electronic device is used by multiple users and when a user coughs or touches a facial feature (e.g., a nose or mouth) while working on the electronic device, the contamination level may be indicated as high. In another example, when the electronic device is used by multiple users in a public place and when the determined users' behavior does not indicate any contamination of the surface of the electronic device, the contamination level may be indicated as medium. In yet another example, when the electronic device is used by multiple users in a home location and when the determined users' behavior does not indicate any contamination of the surface of the electronic device, the contamination level may be indicated as low.

Instructions 110 may be executed by processor 102 to send an alert notification to the electronic device based on the touch-related contamination state. In an example, the alert notification may include a recommended action corresponding to the touch-related contamination state to clean the surface of the electronic device. The recommended action may depend on the contaminated area and the contamination level. In an example, a set of recommended actions (e.g., recommended cleaning approaches and measures) can be configured in computing device 100 (e.g., in a storage device associated with computing device 100). Further, the set of recommended actions may be mapped to different contamination levels (e.g., cleanliness factors) based on a rule-based approach, for instance. Further, the recommended action corresponding to the determined contamination level may be retrieved from the set of recommended actions and sent to the electronic device. In this example, the contamination level and/or the set of recommended actions may be displayed on a user interface (e.g., a display device) of the electronic device.

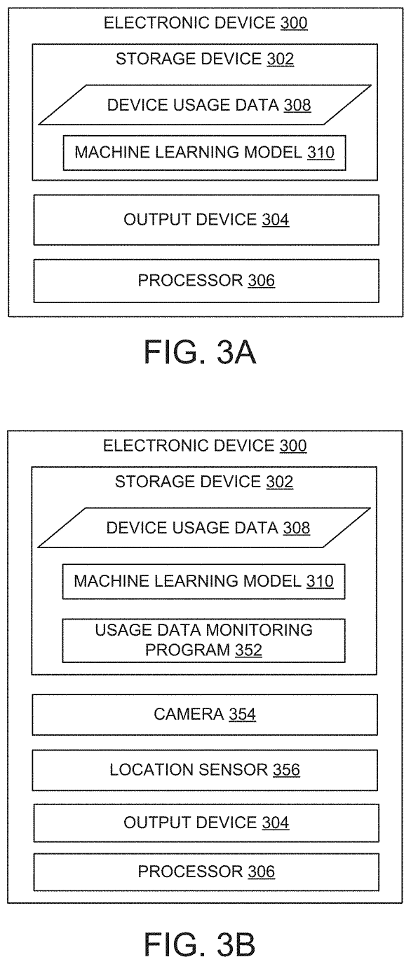

FIG. 2 is a block diagram of an example computing device 200 including a non-transitory machine-readable storage medium 204, storing instructions to determine a machine learning model from a set of machine learning models to estimate a touch-related contamination state. Computing device 200 may include a processor 202 and machine-readable storage medium 204 communicatively coupled through a system bus. Processor 202 may be any type of central processing unit (CPU), microprocessor, or processing logic that interprets and executes machine-readable instructions stored in machine-readable storage medium 204.

Machine-readable storage medium 204 may be a random-access memory (RAM) or another type of dynamic storage device that may store information and machine-readable instructions that may be executed by processor 202. For example, machine-readable storage medium 204 may be synchronous DRAM (SDRAM), double data rate (DDR), Rambus® DRAM (RDRAM), Rambus® RAM, and the like, or storage memory media such as a floppy disk, a hard disk, a CD-ROM, a DVD, a pen drive, and the like. In an example, machine-readable storage medium 204 may be a non-transitory machine-readable medium, where the term “non-transitory” does not encompass transitory propagating signals. In an example, machine-readable storage medium 204 may be remote but accessible to computing device 200.

Machine-readable storage medium 204 may store instructions 206-214. In an example, instructions 206 may be executed by processor 202 to obtain historical device usage data associated with an electronic device. In this example, the historical device usage data may be obtained over a period. For example, the historical device usage data may include user login data, input device usage data, location data, user facial recognition data, user behavior data, or any combination thereof. Further, the input device usage data may include mouse usage data, touchpad usage data, touchscreen usage data, keyboard usage data, or any combination thereof.

Instructions 208 may be executed by processor 202 to process the historical device usage data to generate a train data set and a test data set. Instructions 210 may be executed by processor 202 to train a set of machine learning models, based on the train data set, to estimate a touch-related contamination state of a surface of the electronic device. In an example, instructions to train the set of machine learning models may include instructions to train the set of machine learning models to estimate the touch-related contamination state of a surface of an input device associated with the electronic device. For example, the input device may include a keyboard, a mouse, a touchpad, a touchscreen, or any combination thereof.

In an example, instructions to train the set of machine learning models may include instructions to:
determine, from the processed historical device usage data, a set of features (e.g., individual independent variables) capable of being used to train the set of machine learning models to estimate the touch-related contamination state, and
train the set of machine learning models using the set of features or a subset of the set of features to estimate the touch-related contamination state.

Instructions 212 may be executed by processor 202 to test the trained set of machine learning models with the test data set. In an example, prior to testing the trained set of machine learning models, machine-readable storage medium 204 may store instructions to validate the trained machine learning models to tune an accuracy of the trained machine learning models based on a validation data set of the processed historical device usage data. Thus, a feedback mechanism through the test data set and the validation data set can be built to confirm the correctness of the machine learning models and fine tune the accuracy of the machine learning models, respectively.

Instructions 214 may be executed by processor 202 to determine a machine learning model, for instance, having a high accuracy from the set of trained and tested machine learning models to estimate the touch-related contamination state of the electronic device for real-time device usage data.

Further, machine-readable storage medium 204 may store instructions to:
receive the real-time device usage data associated with the electronic device. In an example, instructions to receive the real-time device usage data associated with the electronic device may include instructions to receive, via an API call, the real-time device usage data from the electronic device at a periodic interval, in response to a user login event to the electronic device, or the like.
estimate the touch-related contamination state of the electronic device by analyzing the real-time device usage data using the determined machine learning model.
generate an alert notification based on the touch-related contamination state.
send the alert notification to the electronic device.

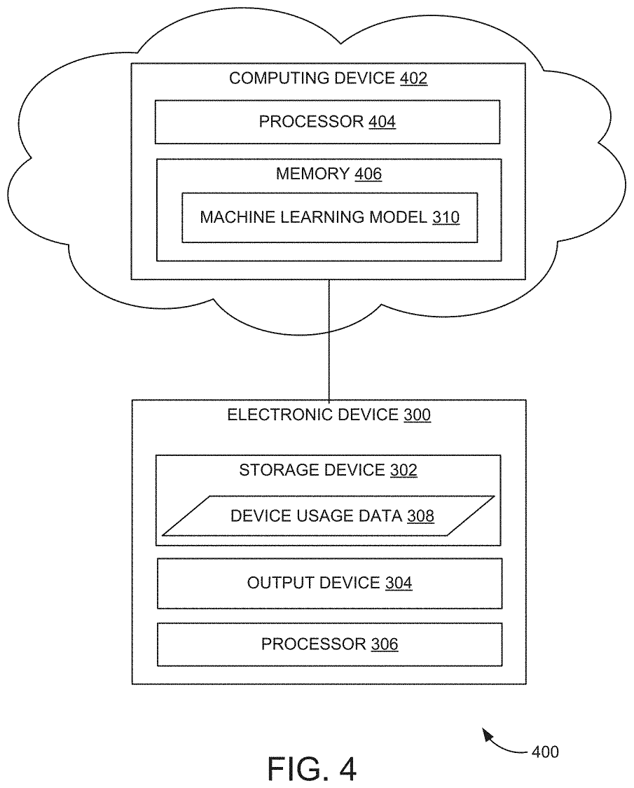

FIG. 3A is a block diagram of an example electronic device 300, including a processor 306 to output an alert notification including a recommended action to clean electronic device 300. Example electronic device 300 may include a desktop computer, a notebook computer, a tablet computer, a smartphone, or the like.

As shown in FIG. 3A, electronic device 300 may include a storage device 302, an output device 304, and processor 306. Storage device 302 may store device usage data 308 associated with an operation of electronic device 300. Further, electronic device 300 may include a machine learning model 310 that can be stored in storage device 302. In other examples, device usage data 308 and machine learning model 310 can be stored in separate storage devices associated with electronic device 300.

In an example, machine leaning model 310 may be trained and tested using historical device usage data to determine the touch-related contamination state of a surface of electronic device 300. Further, machine leaning model 310 may be trained and tested using the historical device usage data to recommend an action corresponding to the determined touch-related contamination state. In such examples, machine leaning model 310 may be trained and tested in a server (e.g., a cloud-based server, Software as a Service (SaaS)-based server, or the like). Further, electronic device 300 may receive trained and tested machine leaning model 310 from the server.

During operation, processor 306 may retrieve stored device usage data 308 for a period in response to receiving a trigger event (e.g., a user login event). Further, processor 306 may apply machine learning model 310 to device usage data 308 to:
detect a change of a user of electronic device 300. In an example, processor 306 may detect the change of the user of electronic device 300 based on user login data used to login to electronic device 300, user facial recognition data captured via a camera associated with electronic device 300, or a combination thereof.
determine a touch-related contamination state of a surface of electronic device 300 in response to the detection. In an example, the touch-related contamination state may include information indicating a contaminated area on the surface of electronic device 300, a contamination level of the contaminated area, or a combination thereof.
determine a recommended action based on the touch-related contamination state.

Further, processor 306 may output an alert notification including the recommended action to clean electronic device 300 via output device 304. In an example, processor 306 may output the alert notification via a defined policy. An example alert notification may include, but not limited to, a visual alert outputted via a visual output device (e.g., a display device), an audible alert outputted via a speaker, a haptic alert outputted via a tactile feedback device (e.g., a vibrator), data that can be sent via a communication interface to an external monitoring device, or any combination thereof.

FIG. 3B is a block diagram of example electronic device 300 of FIG. 3A, depicting additional features. For example, similarly named elements of FIG. 3B may be similar in structure and/or function to elements described with respect to FIG. 3A. As shown in FIG. 3B, electronic device 300 may include a usage data monitoring program 352, a camera 354, and a location sensor 356. For example, device usage data 308 may include user login data and input device usage data. The user login data and the input device usage data may be recorded via usage data monitoring program 352 (e.g., a keylogger program). Further, device usage data 308 may include location data obtained via location sensor 356 (e.g., a GPS sensor). Furthermore, device usage data 308 may include user facial recognition data and user behavior data obtained via camera 354.

In some examples, the functionalities described in FIG. 3B, in relation to instructions to implement functions of usage data monitoring program 352, and any additional instructions described herein in relation to the storage medium, may be implemented as engines or modules including any combination of hardware and programming to implement the functionalities of the modules or engines described herein. The functions of usage data monitoring program 352 may also be implemented by a processor.

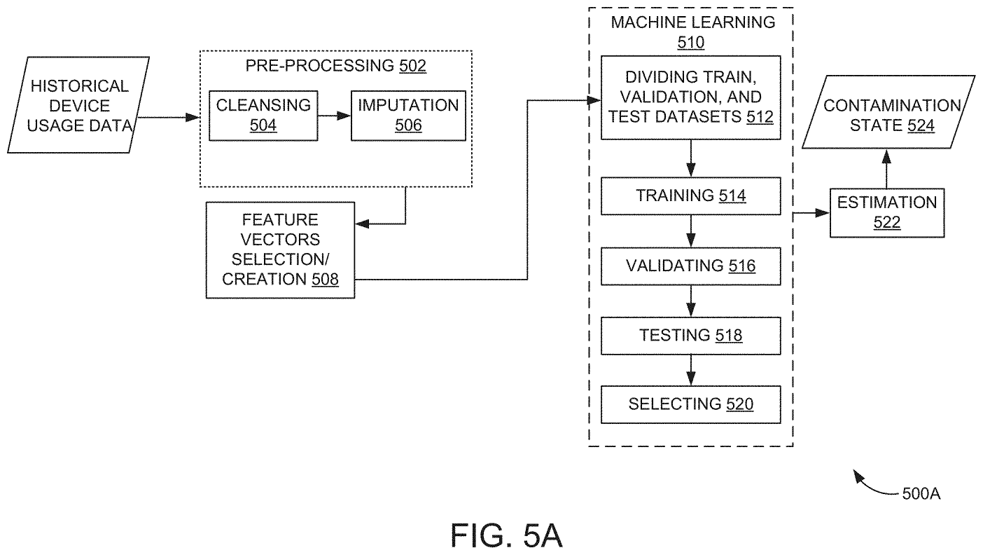

FIG. 4 is a block diagram of an example computing environment 400, depicting example electronic device 300 of FIG. 3A in communication with a computing device 402. For example, similarly named elements of FIG. 4 may be similar in structure and/or function to elements described with respect to FIG. 3A. In the example shown in FIG. 4, machine learning model 310 may be implemented as part of computing device 402. Example computing device 402 may be a cloud-based server. Further, computing device 402 may include a processor 404 and a memory 406. In the example shown in FIG. 4, memory 406 may include machine learning model 310.

During operation, computing device 402 may receive device usage data 308 from electronic device 300. Further, processor 404 may apply machine learning model 310 to received device usage data 308 to detect a change of a user of electronic device 300 and, in response to the detection, determine a touch-related contamination state of a surface of electronic device 300. Furthermore, processor 404 may determine a recommended action based on the touch-related contamination state. Upon determining the recommended action, processor 404 may send an alert notification including the recommended action to electronic device 300. In this example, processor 306 of electronic device 300 may receive the alert notification and output the alert notification via output device 304.

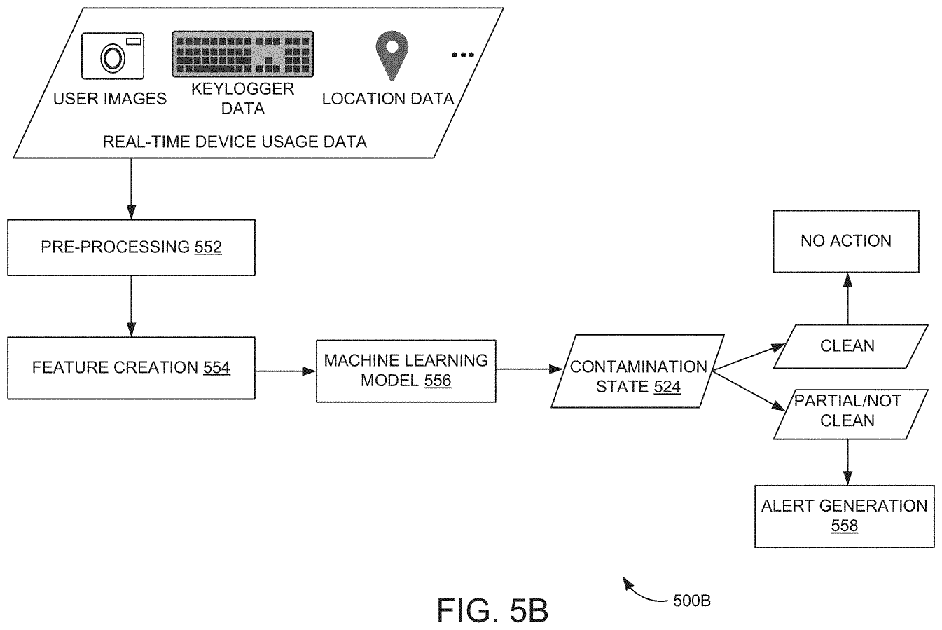

FIG. 5A is a flowchart illustrating an example process 500A for training a machine learning model based on historical device usage data. In one example, process 500A may be implemented in a computing device (e.g., a cloud-based server). At 502, historical device usage data associated with an electronic device may be received from the electronic device. For example, audit log event monitoring program and a keylogger program running in the electronic device may provide information regarding user login and associated work pattern, respectively. In another example, a location sensor in the electronic device may provide location information. The location information may provide information regarding a location (e.g., a public or a private space) of the electronic device.

In yet another example, a camera of the electronic device may provide user images that can be used to differentiate users and to categorize the device usage data per user. Further at 502, the historical device usage data may be pre-processed. In one example, pre-processing the historical device usage data may include cleansing the data (e.g., at 504), imputing the data (e.g., at 506), or any combination thereof.

In an example, cleansing the data may include detecting and replacing an outlier value of a variable in the historical device usage data. In another example, cleansing the data may include normalizing a value of a variable in the historical device usage data. Further, the historical device usage data may be imputed for any missing data value, invalid data value, or scaling a data value. In this example, missing or invalid data values can be processed to impute values to replace the missing or invalid data values. In other words, the historical device usage data may be imputed to insert estimates for missing values that may have minimal impact on the analysis method. The historical device usage data may be imputed through different statistical processes such as mean, previous entry, next entry, automated method (e.g., mice in R), and the like.

Further, at 508, a set of features (e.g., feature vectors) with a plurality of parameters (e.g., that are capable of being used to train the set of machine learning models) may be selected or generated from the pre-processed historical device usage data. At 510, a machine learning model may be built with the cleansed and imputed data with the selected feature vectors. In an example, the machine learning model may be built as described in blocks 512 to 520. At 512, the cleansed and imputed data may be divided into training data, validation data, and test data. For example, the cleansed and imputed data may be divided into 60% of training data, 20% of validation data, and 20% of test data. In other words, first 60% entries may be provided as the training data, next 20% entries may be provided as the validation data, and last 20% entries may be provided as the test data.

At 514, multiple machine learning models may be built with 60% training data. At 516, the machine learning models may be validated with 20% validation data. In one example, the machine learning models may be tuned based on the validation. At 518, upon validating the machine learning models, the machine learning models may be tested with 20% test data.

At 520, a machine learning model having a high accuracy may be selected from the trained and tested machine learning models. In some examples, the selected machine learning model can be stored in a low latency database. The low latency database may facilitate in querying of the stored machine learning model with minimal delay (i.e., minimum latency), for instance, via a representational state transfer API (REST API). At 522, the selected machine learning model can be applied on real-time device usage data received from the electronic device to estimate a touch-related contamination state 524 of the electronic device. An example process to estimate touch-related contamination state 524 of the electronic device is described in FIG. 5B.

FIG. 5B is a flowchart illustrating an example process 500B for determining touch-related contamination state 524 of the electronic device by applying the trained machine learning model (e.g., of FIG. 5A) on real-time device usage data. In the example shown in FIG. 5B, process 500B may be implemented in a computing device (e.g., a cloud-based server). At 552, real-time device usage data for a period may be received from the electronic device and the received real-time device usage data may be pre-processed. In one example, pre-processing the real-time device usage data may include cleansing the data, imputing the data, or any combination thereof. Example real-time device usage data is depicted below in table 1.

TABLE 1

| | Audit | | Location | | Device | No. of | | Changing |
|---|---|---|---|---|---|---|---|---|
| Health | log | Key- | Movement | Device | shut- | user | Changing | screen |
| indicator | event | logger | indicator | age | down | login | brightness | saver |
| 1 | 7 | 37 | 4 | 2 | 6 | 5 | 5 | 5 |
| 1 | 11 | 47 | 8 | 3 | 7 | 3 | 4 | 4 |
| 1 | 9 | 25 | 9 | 4 | 8 | 3 | 5 | 5 |
| 0 | 3 | 7 | 1 | 1 | 4 | 2 | 0 | 0 |
| 0 | 3 | 2 | 2 | 0 | 0 | 2 | 2 | 2 |
| 0 | 1 | 13 | 1 | 0 | 2 | 2 | 1 | 0 |

In the example table 1, the real-time device usage data may include data associated with various parameters such as health indicator, audit log event, keylogger, location movement indicator, device age, device shutdown, number of user logins, changing brightness, changing screen saver, and the like. At 554, a set of features may be created/selected for the pre-processed data. Example features (e.g., auditing log event, keylogger, location movement indicator, and number of user logins) selected from the pre-processed data of table 1 is depicted below in table 2.

TABLE 2

| Auditing | | Location movement | No. of |
|---|---|---|---|
| log event | Keylogger | indicator | user login |
| 7 | 37 | 4 | 5 |
| 11 | 47 | 8 | 3 |
| 9 | 25 | 9 | 3 |
| 3 | 7 | 1 | 2 |

At 556, the machine learning model, as selected at block 520 of FIG. 5A, may be applied to the device usage data to:
determine touch-related contamination state 524 of a surface of the electronic device, and
determine a recommended action based on touch-related contamination state 524.

In an example, when touch-related contamination state 524 of the electronic device is determined as partially clean or not dean, at 558, an alert notification including the recommended action to clean the electronic device may be generated and sent to the electronic device. In another example, when touch-related contamination state 524 of the electronic device is determined as clean, no action may be initiated.

In the examples described herein, the electronic device may make a REST API call at frequent intervals or at a new user login to the electronic device. Upon receiving the REST API call, the computing device may obtain the real-time device usage data from the electronic device. Further, the computing device may communicate with the low latency database, via an API call, for estimating touch-related contamination state 524. Furthermore, the estimated results with suggested actions and measures to clean the electronic device obtained from the low-latency database may be prompted back to the electronic device.

Thus, examples described herein may build an artificial intelligence driven alert mechanism to predict and prompt users to clean the electronic device to ensure healthy living and also to prevent spreading of infectious diseases. Examples described herein may also enhance user experience.

It should be understood that the processes depicted in FIGS. 5A and 5B represent generalized illustrations, and that other processes may be added, or existing processes may be removed, modified, or rearranged without departing from the scope and spirit of the present application. In addition, it should be understood that the processes may represent instructions stored on a computer-readable storage medium that, when executed, may cause a processor to respond, to perform actions, to change states, and/or to make decisions. Alternatively, the processes may represent functions and/or actions performed by functionally equivalent circuits like analog circuits, digital signal processing circuits, application specific integrated circuits (ASICs), or other hardware components associated with the system. Furthermore, the flow charts are not intended to limit the implementation of the present application, but rather the flow charts illustrate functional information to design/fabricate circuits, generate machine-readable instructions, or use a combination of hardware and machine-readable instructions to perform the illustrated processes.

The above-described examples are for the purpose of illustration. Although the above examples have been described in conjunction with example implementations thereof, numerous modifications may be possible without materially departing from the teachings of the subject matter described herein. Other substitutions, modifications, and changes may be made without departing from the spirit of the subject matter. Also, the features disclosed in this specification (including any accompanying claims, abstract, and drawings), and/or any method or process so disclosed, may be combined in any combination, except combinations where some of such features are mutually exclusive.

The terms “include,” “have,” and variations thereof, as used herein, have the same meaning as the term “comprise” or appropriate variation thereof. Furthermore, the term “based on”, as used herein, means “based at least in part on.” Thus, a feature that is described as based on some stimulus can be based on the stimulus or a combination of stimuli including the stimulus. In addition, the terms “first” and “second” are used to identify individual elements and may not meant to designate an order or number of those elements.

The present description has been shown and described with reference to the foregoing examples. It is understood, however, that other forms, details, and examples can be made without departing from the spirit and scope of the present subject matter that is defined in the following claims.

What is claimed is:

1. A non-transitory machine-readable storage medium encoded with instructions that, when executed by a processor of a computing device, cause the processor to:
receive device usage data associated with an electronic device;
determine a touch-related contamination state of a surface of the electronic device by applying a machine learning model to the device usage data; and
send an alert notification to the electronic device based on the touch-related contamination state.

2. The non-transitory machine-readable storage medium of claim 1, wherein the device usage data comprises user login data, input device usage data, location data, user facial recognition data, user behavior data, or any combination thereof.

3. The non-transitory machine-readable storage medium of claim 1, wherein instructions to receive the device usage data comprise instructions to:
receive the device usage data from the electronic device via an application programming interface (API) call.

4. The non-transitory machine-readable storage medium of claim 1, wherein instructions to receive the device usage data comprise instructions to:
receive the device usage data associated with the electronic device at a periodic interval or in response to a user login event to the electronic device.

5. The non-transitory machine-readable storage medium of claim 1, wherein the alert notification is to include a recommended action corresponding to the touch-related contamination state to clean the surface of the electronic device.

6. A non-transitory machine-readable storage medium encoded with instructions that, when executed by a processor of a computing device, cause the processor to:
obtain historical device usage data associated with an electronic device;
process the historical device usage data to generate a train data set and a test data set;
train a set of machine learning models, based on the train data set, to estimate a touch-related contamination state of a surface of the electronic device;
test the trained set of machine learning models with the test data set; and
determine a machine learning model from the set of trained and tested machine learning models to estimate the touch-related contamination state of the electronic device for real-time device usage data.

7. The non-transitory machine-readable storage medium of claim 6, further comprising instructions to:
receive the real-time device usage data associated with the electronic device;
estimate the touch-related contamination state of the electronic device by analyzing the real-time device usage data using the determined machine learning model;
generate an alert notification based on the touch-related contamination state; and
send the alert notification to the electronic device.

8. The non-transitory machine-readable storage medium of claim 7, wherein instructions to receive the real-time device usage data associated with the electronic device comprise instructions to:
receive, via an application programming interface (API) call, the real-time device usage data from the electronic device at a periodic interval or in response to a user login event to the electronic device.

9. The non-transitory machine-readable storage medium of claim 6, wherein instructions to train the set of machine learning models comprise instructions to:
train the set of machine learning models to estimate the touch-related contamination state of a surface of an input device associated with the electronic device, wherein the input device comprises a keyboard, a mouse, a touchpad, a touchscreen, or any combination thereof.

10. The non-transitory machine-readable storage medium of claim 6, wherein the historical device usage data comprises user login data, input device usage data, location data, user facial recognition data, user behavior data, or any combination thereof, and wherein the input device usage data comprises mouse usage data, touchpad usage data, touchscreen usage data, keyboard usage data, or any combination thereof.

11. The non-transitory machine-readable storage medium of claim 6, further comprising instructions to:
prior to testing the trained set of machine learning models, validate the trained machine learning models to tune an accuracy of the trained machine learning models based on a validation data set of the processed historical device usage data.

12. The non-transitory machine-readable storage medium of claim 6, wherein instructions to train the set of machine learning models comprise instructions to:
determine, from the processed historical device usage data, a set of features capable of being used to train the set of machine learning models to estimate the touch-related contamination state; and
train the set of machine learning models using the set of features or a subset of the set of features to estimate the touch-related contamination state.

13. An electronic device comprising:
a storage device;
an output device; and
a processor to:
retrieve device usage data for a period in response to receiving a trigger event, wherein the device usage data is stored in the storage device;
apply a machine learning model to the device usage data to:
detect a change of a user of the electronic device;
determine a touch-related contamination state of a surface of the electronic device in response to the detection; and
determine a recommended action based on the touch-related contamination state; and
output an alert notification including the recommended action to clean the electronic device via the output device.

14. The electronic device of claim 13, wherein the processor is to detect the change of the user of the electronic device based on user login data used to login to the electronic device, user facial recognition data captured via a camera associated with the electronic device, or a combination thereof.

15. The electronic device of claim 13, wherein the touch-related contamination state comprises information indicating a contaminated area on the surface of the electronic device, a contamination level of the contaminated area, or a combination thereof.
